(19) United States
(12) Patent Application Publication
(10) Pub. No.: US 2006/0154281 A1
(43) Pub. Date: Jul. 13, 2006

(54) Reaction chamber

(76) Inventors: Peter A. Kahn, Phoenix, AZ (US); Clifford L. Anderson, Tempe, AZ (US)

Correspondence Address: Amersham Biosciences Corp, 800 Centennial Avenue, Piscataway, NJ, 08855, US

(21) Appl. No.: 11/315,391

(22) Filed: Dec. 22, 2005

Related U.S. Application Data
(60) Provisional application No. 60/638,333, filed on Dec. 22, 2004.
(60) Provisional application No. 60/734,951, filed on Nov. 9, 2005.

(51) Int. Cl. C12Q 1/68; G01N 33/53; C12M 1/34

(52) U.S. Cl. 435/6; 435/7.1; 435/287.2

(57) ABSTRACT

The invention describes novel reaction chambers that include a case with at least one opening and a flat bottom flange attached to the first side of a substrate with at least one microarray of materials attached thereto. The case and the substrate are attached through an adhesive layer with at least one perforations such that the at least one microarrays, the at least one perforations and the at least one openings are aligned and forms at least one individual reaction chambers. Methods of using such chambers are disclosed. Also provided are kits including the novel chambers.

### CROSS-REFERENCE TO RELATED APPLICATIONS

This application claims priority to U.S. provisional patent application No. 60/638,333 filed Dec. 22, 2004 and 60/734,951 filed Nov. 9, 2005; the disclosures of which are incorporated herein by reference in their entireties.

### FIELD OF THE INVENTION

The present invention is directed toward novel reaction chambers, systems and methods of use. More specifically, the invention is directed to novel chambers for use with microarray systems. Methods of using such chambers are also disclosed.

### BACKGROUND OF THE INVENTION

Microarrays have revolutionized biological research over the past decade. As a result, instrumentation for manufacturing and reading spotted microarrays has been widely commercialized. The initial technology for spotting cDNA has now been extended to include spotting other materials, including small molecules, oligonucleotides, proteins (e.g., enzymes, antibodies, etc.), whole cells, and tissue specimens. To a large degree, a standard format has been adopted by the industry: microarrays are manufactured on 25 mm by 75 mm glass slides that are 1 mm thick.

Traditionally, microarrays are processed by washing them with a single sample at a time. It is routine now that people study the interaction of many different samples with a given microarray of materials. For example, one may want to screen thousands of different serum samples from patients with a microarray of 100 different antibodies. Or, one may wish to screen multiple patients for their ability to metabolize a certain drug compound in a microarray of 100 different pairs of single nucleotide polymorphisms (SNPs). For these studies, each of the microarrays on a slide must be separated from the others to avoid cross-contamination from the different samples.

Various apparatus have been designed for partitioning individual microarrays on a slide. For example, U.S. patent application Ser. No. 10/171,128 discloses one such example, although it is quite complex to assemble. Another example is the Lab-Tek II chamber, by NUNC A/S (Denmark). This chamber was designed for use in tissue culturing on a glass slide. This chamber suffers from being a rigid plastic chamber. The slide itself has a special coating to provide adequate adhesion, which would be difficult to implement for bioassay slides. Non-flatness of the rigid chamber would stress the adhesive joint. For scanning with the chamber in place, non-flatness of the chamber would warp the glass, which could adversely affect scanning.

Most, if not all, reactions performed in reaction chambers require mixing of the reaction components. For example amplification of nucleic acid by the polymerase-chain-reaction (PCR) requires mixing DNA template, primers, buffer, polymerase, nucleotides etc. needed for DNA synthesis. Mixing also is required for efficient hybridization of a target nucleic acid to a probe array attached to a surface within a reaction chamber. Simply adding the reaction components separately to a reaction chamber generally does not result in effective mixing. An additional impediment to achieving efficient reaction rates are the minute quantities (e.g. <picomole) of a target analyte obtained in biological samples. Therefore, in the absence of efficient mixing of the reaction components, tens of hours may be required for a detectable result to be obtained.

Recently, high throughput robotics has been developed for biomedical and pharmaceutical research. In this area, instruments are designed to handle microtiter plates. These plates are approximately 85 mm by 125 mm. Wells in these plates are designed with standard spacing. A 96-well plate has twelve columns and eight rows with 9 mm spacing between the centers of adjacent wells. A 384-well plate has twenty-four columns and sixteen rows with 4.5 mm spacing between the centers of adjacent wells. A 1536-well plate has forty-eight columns and thirty-two rows with 2.25 mm spacing between the centers of adjacent wells. Pipetting and plate-washing robots are designed to handle plates of this format.

Thus, there remains a need in the art for devices and methods for more efficient mixing of reaction solutions within a reaction chamber, while at the same time maintaining the consistency and reliability of the reaction, and keeping the device construction relatively simple. There is a need for a device that fits the current glass slide format. There is also a growing need for a device that fits the popular microtiter plate format.

### SUMMARY OF THE INVENTION

The invention describes processes and devices for combining microarrays on substrates with a case containing enclosed wall-structures to form individual reaction chambers. The processes and devices described herein may be adapted for use with microarrays that are arranged on substrates made from a variety of materials. There are also no limitations on the nature of the microarrays or on the shape and dimensions of the substrates. Furthermore, the processes and devices described herein may be adapted for use with any number of cases without limitation to the size, shape, and features of the case; or to the materials and methods used to prepare the case.

In general, the invention disclosed herein is a novel packaging approach for microarray assays. The package is comprised of a case containing individual, enclosed wall structures, adhesively attached to the assay substrate in such a way that the individual microarrays are each separated from the other in an individual chamber. The adhesive obviates the need for spring clips. Preferably, the individual, enclosed wall structures are semi-rigid, thin-walled structures, although other, moldable materials are also envisioned. The flexibility of the semi-rigid wall allows adhesion to glass to overcome warp characteristic of molded plastic parts. A substantial interior height above the glass allows for air-interface mixing. The top opening allows easy loading of reaction components and solutions. The current system can be sealed using a sealing strip or plug. When used in high throughput screening, samples present in one well is prevented from diffusing into an adjacent well. It will be appreciated that by combining microarrays with a case design in this manner, the present invention allows the use of current instrumentation for preparing and scanning microarrays, to be combined with the current instrumentation for processing samples in microtiter plates.

In one aspect, the present invention provides assay chambers including at least one reaction chamber, comprising: (a) a substrate having a first surface and a second surface, wherein at least one reaction area is contained on the first surface; (b) an adhesive layer with at least one perforation; and (c) a case having at least one enclosed wall structures and a flat bottom flange, wherein each of the wall structures define a bottom opening, and a top portion opposite each bottom opening, whereas each top portion contains a top opening. The bottom flange of the case is attached to the first surface of the substrate through the adhesive layer such that each of the at least one reaction area, the at least one perforation and the at least one bottom opening are aligned and forms at least one individual reaction chamber. Optionally, the reaction chamber further comprises an identifiable mark, such as a barcode, on the second surface of the substrate in an area outside of the at least one reaction areas. Alternatively, the reaction chamber may include an identifiable mark, such as a barcode, on top of the bottom flange of the case. Preferably, the substrate is comprised of a material selected from the group consisting of ceramic, glass, silicon, and plastic.

In one embodiment, the enclosed wall structure of the case has a substantial height above the substrate to allow air-interface mixing. The outer dimensions of the bottom flange need not cover the whole glass substrate.

In another embodiment, the case has enclosed semi-rigid thin-wall structures, and a flat, thin bottom flange. In addition, each of the top openings are substantially the same size as the corresponding bottom openings, and the flexibility of the semi-rigid thin-wall and the thin bottom flange allows tight adhesion to the substrate and overcomes warp characteristics of the case or the substrate. Preferably, the semi-rigid, thin-walled case is made of plastics. Most preferably, the case is made of polypropylene. Preferably, the thin wall and the thin bottom flange of the case are of substantially the same thickness. Also preferably, the openings of the case have rims to allow easy and secure attachment of a sealing strip. More preferably, the width of the rims is substantially twice the thickness of said thin wall.

In yet another embodiment, the top openings of the case consist of small ports, and the remaining parts of the top portions are enclosed.

In another embodiment, case is made of rubber. When the case is made of rubber, the outer dimensions of the bottom flange could be larger than the glass substrate. This allows the glass to be recessed into the rubber to help protect the corners of the glass slide.

In still another embodiment, the substrate is the same size of a standard microscopic slide. For microarray analysis, each reaction area includes a microarray of material to be analyzed. Any number of microarrays can be manufactured on the substrate. Preferably, these microarrays are evenly spaced across the substrate, and form a symmetrical pattern. The case and the adhesive layer have similar dimensions as the substrate. The case and the adhesive layer have matching patterns as the microarrays such that each microarray is partitioned into an individual chamber, after the formation of these chambers. Commonly the substrate contains 1, 2, 3, 4, 6, 8, 14, 16 or 24 arrays. Although any number of microarrays and any pattern is possible.

Alternatively, the substrate is the same size of a standard microtiter plate. Similar to the substrates of the above embodiment, any number of microarrays can be manufactured on the substrate. Preferably, these microarrays are evenly spaced across the substrate, and form a symmetrical pattern. The case and the adhesive layer have similar dimensions as the substrate. The case and the adhesive layer have matching patterns as the microarrays such that each microarray is partitioned into an individual chamber, after the formation of these chambers. Commonly the substrate contains 96 or 384 arrays, with a layout identical to the pattern of the current microtiter plates on the market. Although any number of microarrays and any pattern is possible.

In one embodiment, the reaction areas contain microarrays of spotted material, selected from small molecules, biomolecules, cells and tissue samples. Specifically, the biomolecules are selected from proteins, polynucleotides, and polysaccharides.

In one embodiment, the adhesive layer is a double-sided adhesive. Preferably, the double-sided adhesive is pressure-sensitive.

In another aspect, the present invention provides an assay system including the at least one reaction chamber, and a top sealing strip.

In one embodiment, a contiguous gap is maintained between the upper inner surface of the sealing strip and a sample fluid within the chamber to allow air-interface mixing.

In another aspect, the present invention provides an assay system including the at least one reaction chamber, and a sealing plug. It is noted that here, the top openings of the reaction chambers consist of small ports, and the remaining parts of said top portions are enclosed.

In one embodiment, a contiguous gap is maintained between the upper inner surface of the sealing plug and a sample fluid within the chamber to allow air-interface mixing.

In another aspect, the present invention provides a method for preparing at least one reaction chamber comprising: providing a substrate with a first surface and a second surface, wherein at least one reaction area is contained on the first surface; providing an adhesive layer with at least one perforations; providing a case having at least one enclosed wall structure and a flat bottom flange, wherein each of the wall structures define a bottom opening, and a top portion opposite each bottom opening, whereas each top portion contains a top opening; adhering the case to a first face of the adhesive layer so that the at least one bottom openings are aligned with the at least one perforations; and adhering the first surface of the substrate to a second face of the adhesive layer so that the at least one reaction areas are aligned with the at least one perforations to form at least one individual reaction chambers.

In another aspect, the present invention provides a method for screening microarrays of materials comprising: preparing at least one reaction chamber containing a microarray, according to the method above; processing the microarrays of materials in the reaction chambers to acquire one or more desired characteristics of the microarray of materials; and scanning the microarrays of materials to identify these characteristics.

### DESCRIPTION OF THE DRAWINGS

FIG. 1A depicts one embodiment of a two assay reaction chamber system 100 having two assay chambers defined by a semi-rigid, thin-walled case 110, perimeter adhesive layer 130, and substrate 150. The thin-walled case has two thin-walled 112 openings, a flat, thin bottom flange 114, and rims 116 at the upper end of the thin-walled openings.

FIG. 1B is an exploded view of FIG. 1A.

FIG. 2 depicts another embodiment of a two assay reaction system 200 having two assay chambers defined by a semi-rigid, thin-walled case 210, perimeter adhesive layer 230, and substrate 250 having arrays 261 and 262. The thin-walled case has two thin-walled 212 openings, a flat, thin bottom flange 214, and rims 216 at the upper end of the thin-walled openings. A top sealing strip 280 covers the opening of the thin-walled case to prevent evaporation of reaction content. Also shown is an optional sheet 290 on top of the bottom flange of the case which may carry an identifiable mark or barcode.

FIG. 2A provides an exploded view of the system.

FIG. 2B provides a top view of the system, while

FIG. 2C and FIG. 2D provides sectional views.

FIG. 3 shows examples of 4, 8, 16 assay reaction chambers according to embodiments of the invention.

FIG. 4 shows an example of an alternative embodiment of the invention. A two assay reaction chamber system is shown. The chambers have enclosed tops, with two open ports each sealed by a plug.

### DETAILED DESCRIPTION OF THE PREFERRED EMBODIMENTS

The present application mentions various patents, scientific articles, and other publications, including US patent application publication US 2003/0064507. The contents of each such item are hereby incorporated by reference.

The invention describes reaction chambers and systems comprising microarrays on substrates and a case with openings. In general, the substrates having microarrays and the case are combined through an adhesive layer in such a way that the individual microarrays end up at the bottom of different chambers formed by the opening, each separated from the other by a water-tight seal. When used in high throughput screening, the water-tight seal prevents samples present in one well from diffusing into an adjacent well. It will be appreciated that by combining microarrays with a case in this manner, the present invention provides improved flexibility for the microarray reaction systems. These chambers and systems can take on a variety of dimensions and formats. Preferably, the individual, enclosed wall structures are semi-rigid, thin-walled structures, although other, moldable materials are also envisioned. The systems provide improved mixing of solutions within the chamber through air-interface mixing, and therefore improved processing and detection of target analytes.

Methods of Preparing a Reaction Chamber

In one aspect, it is provided a method for preparing at least one reaction chamber comprising: providing a substrate with a first surface and a second surface, wherein at least one microarray of materials are attached to the first surface; providing an adhesive layer with at least one perforations; providing a case with at least one openings and a flat thin bottom flange; adhering the case to the first face of the adhesive layer so that the at least one openings are aligned with the at least one perforations; and adhering the first surface of the substrate to the second face of the adhesive layer so that the at least one microarrays are aligned with the at least one perforations. The reaction chambers formed can be sealed by a top sealing strip. Alternatively, when the top openings of the chamber consist of small ports, they can be sealed by a plug as well. Preferably, the case is a semi-rigid, thin-walled case, and the flexibility of the semi-rigid thin wall and the thin bottom flange allows tight adhesion to the substrate and overcomes warp characteristics of the case or the substrate.

The processes and devices described herein may be adapted for use with microarrays that are arranged on substrates made from a variety of materials. There are also no limitations on the nature of the microarrays or on the shape and dimensions of the substrates. In certain embodiments, the substrates may have the dimensions of a standard glass slide, i.e., 25 mm by 75 mm and 1 mm thickness. In other embodiments, the substrates may have the dimensions of a standard microtiter plate, i.e., 85 mm by 125 mm and 1 mm thickness. However, the present invention is in no way limited to rectangular substrates having these dimensions. In preferred embodiments the substrates are rigid meaning that the substrates are solid and do not readily bend, i.e., the substrates are not flexible. As such, rigid substrates are sufficient to provide physical structure to the materials present thereon under the conditions in which the microarray is employed, particularly under high throughput handling conditions. Preferred, but non-limiting, materials are plastic and glass. The microarrays themselves may include a variety of materials such as, but not limited to, small molecules, e.g., from a combinatorial library; biomolecules, e.g., proteins, polynucleotides, and/or carbohydrates; whole cells; and tissue specimens.

Furthermore, the processes and devices described herein may be adapted for use with any type of case without limitation to the size, shape, and features of the case; the size, shape, and number of openings; or to the materials and methods used to prepare the case. The cases are typically made by injection molding, casting, machining, laser cutting, or vacuum sheet forming one or more resins. The cases may be made from transparent or opaque materials. Some cases maybe made of plastics, forming a semi-rigid, thin-walled case. Still others maybe made of flexible, molded material such as rubber or silicone to allow conformity instead of relying on the thin wall structure.

A variety of double-sided adhesives that include acrylic and silicone adhesives are available commercially. The properties of these and other adhesives are described in a variety of commercial manuals, e.g., “3M Designer's Reference Guide to Adhesive Technology” and “3M Manual of Double Coated Tapes, Adhesive Transfer Tapes and Reclosable Fasteners” both from 3M of St. Paul, Minn., see also the adhesives described in “Adhesion and Bonding”, Encyclopedia of Polymer Science and Engineering, Vol. 1, pp. 476-546, Interscience Publishers, 1985.

Preferably, the adhering steps are preceded by a step of aligning the substrates with the case and the adhesive layer. The aligning step may be performed manually or more preferably using an aligning device. Any device that aligns the substrates with the aligning device may include a rigid material with one or more casings that are shaped and dimensioned to accommodate a substrate. According to such embodiments, the substrates are first placed into the one or more casings. The case and adhesive layer are then placed over the substrates so that they adhere.

The Reaction Chamber

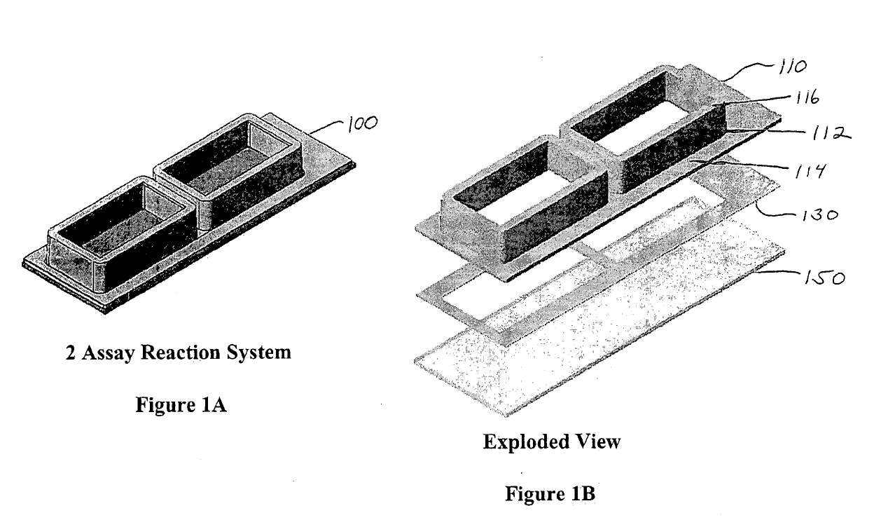

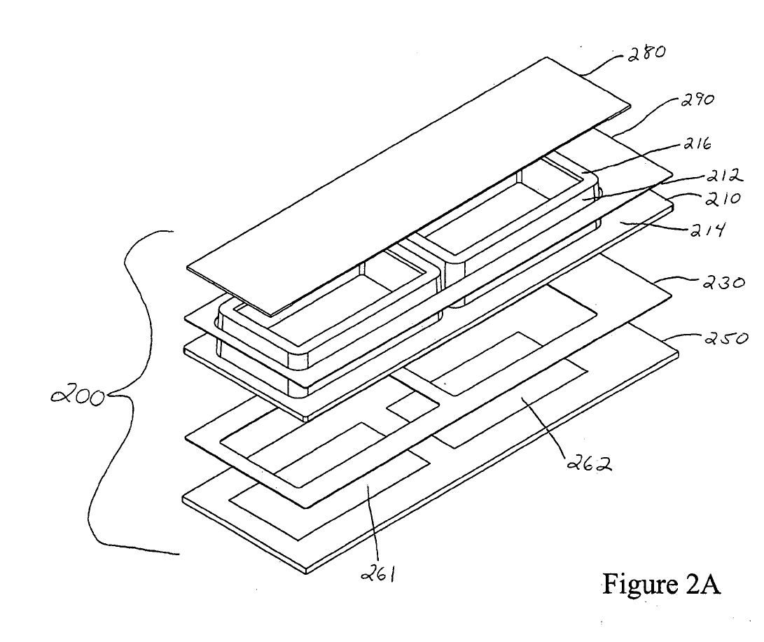

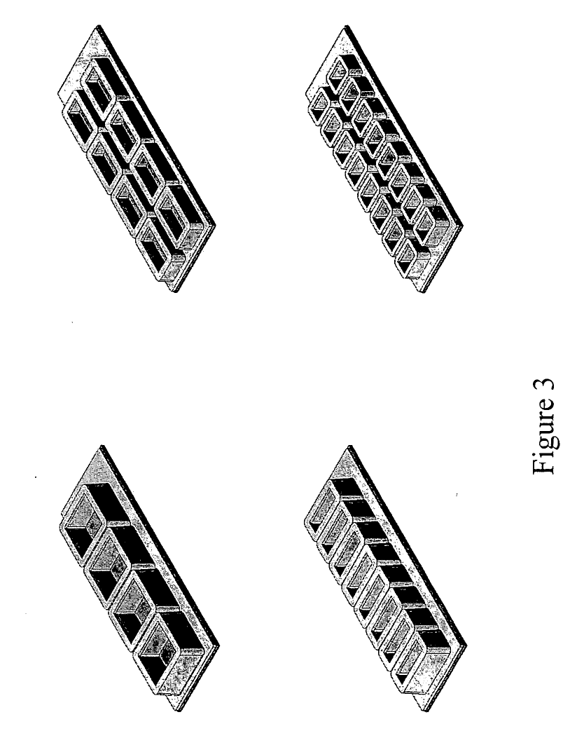

In one aspect, the present invention provides assay chambers including at least one reaction chamber, comprising: (a) a substrate with a first surface and a second surface, wherein at least one reaction area is attached to the first surface; (b) an adhesive layer with at least one perforation; and (c) a case having at least one enclosed wall structures and a flat bottom flange, wherein each of the wall structures define a bottom opening, and a top portion opposite each bottom opening, whereas each top portion contains a top opening. The bottom flange of the case is attached to the first surface of the substrate through the adhesive layer such that each of the at least one reaction area, the at least one perforation and the at least one bottom opening are aligned and forms at least one individual reaction chamber. Optionally, the reaction chamber further comprises an identifiable mark, such as a serial number or a barcode, on the second surface of the substrate in an area outside of the at least one reaction areas. Alternatively, the reaction chamber may also include an identifiable mark, such as a serial number or a barcode, on top of the bottom flange of the case. Optionally, a top sealing strip is provided to seal off the assay chamber. Preferably, the substrate is comprised of a material selected from the group consisting of ceramic, glass, silicon, and plastic. Also preferably, the enclosed wall structure of the case has a substantial height above the substrate to allow air-interface mixing. FIGS. 1-4 provide examples of such assay chamber assemblies.

As shown in FIG. 1, one example of such a case has enclosed semi-rigid thin-wall structures, and a flat, thin bottom flange. In addition, each of the top openings are substantially the same size as the corresponding bottom openings, and the flexibility of the semi-rigid thin-wall and the thin bottom flange allows tight adhesion to the substrate and overcomes warp characteristics of the case or the substrate. Preferably, the semi-rigid, thin-walled case is made of plastics. Most preferably, the case is made of polypropylene. Preferably, the thin wall and the thin bottom flange of the case are of substantially the same thickness. Also preferably, the openings of the case have rims to allow easy and secure attachment of a sealing strip. More preferably, the width of the rims is substantially twice the thickness of said thin wall.

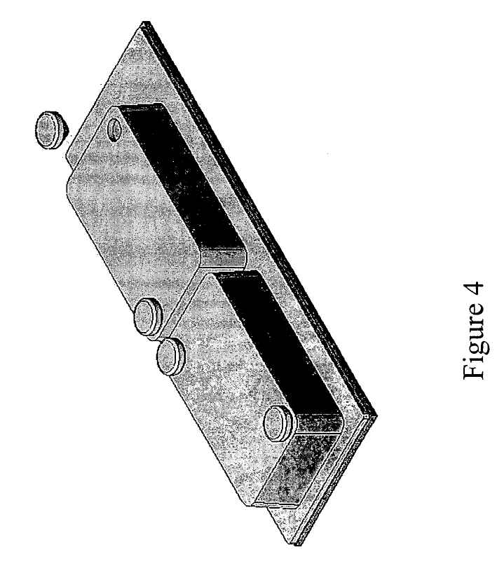

An alternative example of such a case is shown in FIG. 4. Here, the top openings of the case consist of small ports, and the remaining parts of the top portions are enclosed.

The case can also be made of a flexible, molded material such as rubber, or silicone. These materials allow conformity instead of relying on the thin wall structure.

Microarrays

Each reaction area on the substrate may contain a microarray including a variety of materials including but not limited to small molecules, e.g., a combinatorial library; biomolecules, e.g., proteins, polynucleotides, and/or carbohydrates; whole cells; and tissue specimens. The materials are preferably stably associated with the surface of a substrate. By stably associated is meant that the materials maintain their position relative to the substrate under conditions of use, e.g., high throughput screening. As such, the materials can be non-covalently or covalently associated with a substrate surface. Examples of suitable non-covalent associations include non-specific adsorption, specific binding through a specific binding pair member covalently attached to a substrate surface, and entrapment in a matrix material, e.g., a hydrated or dried separation medium. Examples of suitable covalent associations include covalent bonds formed between small molecules or biomolecules and a functional group present on a surface of the substrate, where the functional group may be naturally occurring or present as a member of an introduced linking group, as described in greater detail below.

The substrates of the subject microarrays may be fabricated from a variety of materials. In preferred embodiments the substrate is rigid meaning that the substrate is solid and does not readily bend, i.e., the substrate is not flexible. As such, rigid substrates are sufficient to provide physical structure to the materials present thereon under the conditions in which the microarray is employed, particularly under high throughput handling conditions. Preferably, the materials from which the substrate is fabricated exhibit a low level of non-specific binding of target sample under the conditions of the assay. In many situations, it will also be preferable to employ a material that is transparent to visible and/or UV light. Specific materials of interest include: glass; plastics, e.g., polytetrafluoroethylene, polypropylene, polystyrene, polycarbonate, and blends thereof, and the like; metals, e.g. gold, platinum, and the like; etc.

The substrate of the subject microarrays comprises at least one surface on which microarrays of materials are present, where the surface may be smooth or substantially planar, or have irregularities, such as depressions or elevations. The surface on which the microarrays of materials are presented may be modified with one or more different layers of compounds that serve to modulate the properties of the surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include inorganic and organic layers such as metals, metal oxides, polymers, small organic molecules and the like. Polymeric layers of interest include layers of proteins, polynucleotides or mimetics thereof, e.g., peptide nucleic acids and the like; polysaccharides, phospholipids, polyurethanes, polyesters, polycarbonates, polyureas, polyamides, polyethyleneamines, polyarylene sulfides, polysiloxanes, polyimides, polyacetates, and the like, where the polymers may be hetero- or homopolymeric, and may or may not have separate functional moieties attached thereto, e.g., conjugated.

In certain embodiments the spots within a given microarray include the same material. In other embodiments each spot includes a different material. It is further to be understood that the different microarrays on a particular substrate may be the same or different. Generally, a given substrate may include any number of individual microarrays arranged thereon. The centers of the microarrays are spaced and arranged according to the arrangement of openings of the case. It is to be understood that the substrates need not include a microarray at each and every location on the substrate that corresponds with a perforation and well.

The substrates upon which the subject patterns of materials are preferably presented may take a variety of configurations. Thus, the substrate could have an overall slide or plate configuration, such as a rectangular or disc configuration, where an overall rectangular configuration, as found in standard microarrays and microscope slides, is preferred. For example, the length of the substrates may be at least about 10 mm and may be as great as 400 mm or more, but usually does not exceed about 300 mm and may often not exceed about 150 mm. The width of the substrate may be at least about 10 mm and may be as great as 300 mm, but usually does not exceed 200 mm and often does not exceed 100 mm. The thickness of the substrate will generally range from 0.01 mm to 10 mm, depending at least in part on the material from which the substrate is fabricated and the thickness of the material required to provide the requisite rigidity. In certain preferred embodiments, the substrate is a 25 mm by 75 mm glass slide that is about 1 mm thick. In other embodiments, the substrates may have the dimensions of a standard microtiter plate, i.e., 85 mm by 125 mm and 1 mm thickness. However, the present invention is in no way limited to rectangular substrates having these dimensions.

Substrates that include a variety of microarrays of materials arranged thereon are available commercially. Furthermore, a variety of methods for preparing microarrays of small molecules, biomolecules, whole cells, and tissue samples are known in the art. In particular, in addition to the well known techniques for preparing microarrays of polynucleotides, a variety of techniques have recently been developed that enable small molecules, proteins, carbohydrates, whole cells, and tissue samples to be microarrayed on the surface of substrates such as glass and plastic slides.

Adhesive Layer

By “adhesion layer”, “adhesive layer”, and grammatical equivalents herein are meant a substance or compound that adheres a case and substrate of a reaction system together to both provide a reaction chamber and to a provide a seal that substantially prevents leakage of the contents of the chamber. As will be appreciated by those in the art this may take on a variety of different forms. In one embodiment, adhesives are used to attach the case to the substrate. Examples of adhesives include a double-sided sheet, rubber adhesives, and liquid adhesives, such as silicon, acrylic, and combinations thereof. Desirable characteristics of the adhesive is that it provide sufficient adhesive strength between layers, and optionally that it can be cleanly removed from a substrate. For example, in one embodiment the adhesive comprises a UV release adhesive having a high tack in the absence of UV light but has a low tack after exposure to UV light. Preferably, the array is masked during UV light exposure. Thus, the substrate may be conveniently removed from the other chamber components following UV exposure and the array is easily scanned.

The Case

In one aspect, the present invention provides a case for forming a reaction chamber including: a wall structure with at least one opening and a flat thin bottom flange; whereby the bottom flange of the case can be attached to a first surface of the substrate, by an adhesive layer, to form at least one individual reaction chambers. Optionally, the case may also include an identifiable mark, such as a barcode, on top of the bottom flange of the case. The case preferably contains semi-rigid thin wall structures and thin bottom flange, allowing tight adhesion to the substrate that overcomes warp characteristics of the case or the substrate.

Preferably, the semi-rigid, thin-walled case is made of plastics. Most preferably, the semi-rigid, thin-walled case is made of polypropylene. Alternatively, the case maybe made of flexible, molded material such as rubber or silicone to allow conformity instead of relying on the thin wall structure. The top portion of the case optionally contains small ports, with the remaining parts closed. The ports may be sealed by a sealing strip, or a plug (FIG. 4). A case with small ports offered an advantage in some instances by reducing splash and cross-contamination among the different chambers during analyte loading. The device of the present invention may include any type of case without limitation to the size, shape, and features of the plate; the size, shape, and number of openings; or to the materials and methods used to prepare the plate. The cases are typically made by injection molding, casting, machining, laser cutting, or vacuum sheet forming one or more resins. The cases may be made from transparent or opaque materials.

The case and the adhesive layer have matching patterns as the microarrays on the substrate, such that each microarray is partitioned into an individual chamber, after the formation of these chambers. Any number of microarrays can be manufactured on the substrate. Preferably, these microarrays are evenly spaced across the substrate, and form a symmetrical pattern. When the substrate has the same dimensions of a standard microscopic slide, commonly the substrate contains 1, 2, 3, 4, 6, 8, 12, 14, 16, or 24 arrays. When the substrate has the same dimensions of a standard microtiter plate, commonly the substrate contains 96 or 384 arrays, with a layout identical to the pattern of the current microtiter plates on the market. Although any number of microarrays and any pattern is possible.

Plastic and glass parts are difficult to keep flat during the manufacturing processes. The current design overcomes this problem by providing a chamber that is designed with “thin walls” or flexible materials that allow the case conform to the substrate surface during assembly. The wall thickness in the first realization of this part used wall thickness of less than 1 mm. One design of a polypropylene case has a thin wall of about 0.9 mm thick, which puts it at slightly less than the glass thickness. This gives the case a desired “semi-rigid” characteristic. The soda-lime glass used for CodeLink™ bioarrays has a modulus of elasticity of Es=70˜73 GPa. Polypropylene has Ec=1.1˜1.4 GPa (chamber case modulus). For flat plate geometry, such as the substrate and the flanges of the chamber case, the relative stiffness is then related by (tf/ts)ˆ3*(Ec/Es), where ts=substrate thickness, tf=flange thickness. For the thicknesses and moduli indicated, this results in relative stiffness Sf/Ss=0.013. Thus, the flange is substantially less rigid than the substrate. Even thinner chamber case was made with materials made from PETG. The added advantage was the ability to use a lower cost thermoforming process to mold the chambers.

Preferably, the thin wall and the thin bottom flange of the case are of substantially the same thickness. Also preferably, the openings of the case have rims to allow easy and secure attachment of a sealing strip. Most, preferably, the width of the rims is substantially twice the thickness of said thin wall.

Air-Interface Mixing

By “mixing” and grammatical equivalents herein are meant to circulate or agitate a fluid such that at least one substance in the fluid is distributed, preferably but not required to be, evenly within an area or a volume. Accordingly, mixing includes, for example, the circulation or agitation of a fluid, causing a more even distribution of at least one substance, whether particulate, dissolved or suspended, in the fluid. Within the definition of mixing also is contemplated the continued circulation or agitation of a fluid, even though the continued mixing does not further distribute a substance within the fluid. Thus, in a preferred embodiment, mixing results in a fluid that is spatially homogeneous or uniform. The degree of mixing, the timing and the force applied to effectuate the mixing are selected at the discretion of the practitioner based on the target analyte, the sample, the detection method etc. as known in the art.

The wall of the case has a substantial height above the substrate. During an assay, a contiguous gap is maintained between the upper inner surface of the sealing strip and a sample fluid within the chamber to allow air-interface mixing. Mixing occurs in the presence of air in the chamber. For example, a contiguous gap may be employed for mixing. Without being bound by theory, the contiguous gap permits displacement of the fluid within the chamber resulting in mixing.

Methods of Screening Microarrays Using the Disclosed Device

The present invention also provides methods of screening microarrays using the devices described herein. These methods include: providing a substrate with at least one microarrays, an adhesive layer with at least one perforations, and a case with at least one openings as described hereinabove; adhering the case to the first face of the adhesive layer so that the at least one openings are aligned with the at least one perforations; and adhering the first surface of the substrate to the second face of the adhesive layer so that the at least one microarrays are aligned with the at least one perforations; whereby the at least one microarray, the at least one perforation and the at least one opening forms at least one individual reaction chambers; processing the microarrays of materials in the reaction chambers to determine one or more desired characteristics of the materials; and scanning the microarrays of materials to identify the characteristics.

In certain embodiments, the substrate is removed from the device before scanning the microarrays of materials. In other embodiments, the substrate is not removed from the device before scanning the microarrays of materials.

The devices of the invention are used to detect target analytes. “Target analyte” and grammatical equivalents herein are used to refer to analytes to be detected or quantified. “Contamination analyte” and grammatical equivalents herein are used to refer to analytes present in a sample that are not to be detected. These “contamination analytes” frequently interfere with the efficient detection of “target analytes”. Target analytes preferably bind to a binding ligand, as is more fully described below.

Target analytes may be present in any number of different sample types, including, but not limited to, bodily fluids including blood, lymph, saliva, vaginal and anal secretions, urine, feces, perspiration and tears, and solid tissues, including liver, spleen, bone marrow, lung, muscle, brain, etc. and environmental samples, such as, soil, water, air, plants, and the like; and manufactured products, etc.

As will be appreciated by those in the art, a large number of target analytes may be manipulated and subsequently detected using the present methods; basically, any target analyte for which a binding ligand, described herein, may be made may be detected using the methods of the invention.

Suitable target analytes include organic and inorganic molecules, including biomolecules. In a preferred embodiment, the target analyte may be an environmental pollutant (including pesticides, insecticides, toxins, etc.); a chemical (including solvents, polymers, organic materials, etc.); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc); whole cells (including prokaryotic (such as pathogenic bacteria) and eukaryotic cells, including mammalian tumor cells); viruses (including retroviruses, herpesviruses, adenoviruses, lentiviruses, etc.); and spores; etc. Particularly preferred target analytes are environmental pollutants; nucleic acids; proteins (including enzymes, antibodies, antigens, growth factors, cytokines, etc); therapeutic and abused drugs; cells; and viruses.

In a preferred embodiment, the target analyte is a nucleic acid.

In a preferred embodiment, the present invention provides methods of manipulating and detecting target nucleic acids. By “target nucleic acid” or “target sequence” or grammatical equivalents herein means a nucleic acid sequence on a single strand of nucleic acid. The target sequence may be a portion of a gene, a regulatory sequence, genomic DNA, cDNA, RNA including mRNA and rRNA, or others. It may be any length, with the understanding that longer sequences are more specific. In some embodiments, it may be desirable to fragment or cleave the sample nucleic acid into fragments of 100 to 10,000 base pairs, with fragments of roughly 500 base pairs being preferred in some embodiments. As will be appreciated by those in the art, the complementary target sequence may take many forms. For example, it may be contained within a larger nucleic acid sequence, e.g. all or part of a gene or mRNA, a restriction fragment of a plasmid or genomic DNA, among others.

Probes (including primers) are made to hybridize to target sequences to determine the presence or absence of the target sequence in a sample. Generally speaking, this term will be understood by those skilled in the art.

In a preferred embodiment, the target analyte is a protein. As will be appreciated by those in the art, there are a large number of possible proteinaceous target analytes that may be detected using the present invention.

In addition, any of the molecules for which antibodies may be detected may be detected directly as well; that is, detection of virus or bacterial cells, therapeutic and abused drugs, etc., may be done directly.

Suitable analytes include carbohydrates, including but not limited to, markers for breast cancer (CA15-3, CA 549, CA 27.29), mucin-like carcinoma associated antigen (MCA), ovarian cancer (CA125), pancreatic cancer (DE-PAN-2), prostate cancer (PSA), CEA, and colorectal and pancreatic cancer (CA 19, CA 50, CA242).

In another aspect, the present invention provides a kit comprising: a substrate with a first surface and a second surface, wherein at least one microarray of materials are attached to the first surface; an adhesive seal with at least one perforation; and a case with at least one opening and a flat bottom flange.

In yet another aspect, the present invention provides a kit comprising: an adhesive seal with at least one perforation; and a case with at least one opening and a flat bottom flange, wherein the dimensions of the bottom flange substantially equal the size of a substrate.

The following example is offered for purpose of illustration, not limitation.

### EXAMPLE 1

One example is illustrated in FIG. 1A, with an exploded view in FIG. 1B. The two assay reaction chamber system 100 has two assay chambers defined by a semi-rigid, thin-walled case 110, perimeter adhesive layer 130, and substrate 150. The thin-walled case has two thin-walled 112 openings, a flat, thin bottom flange 114, and rims 116 at the upper end of the thin-walled openings.

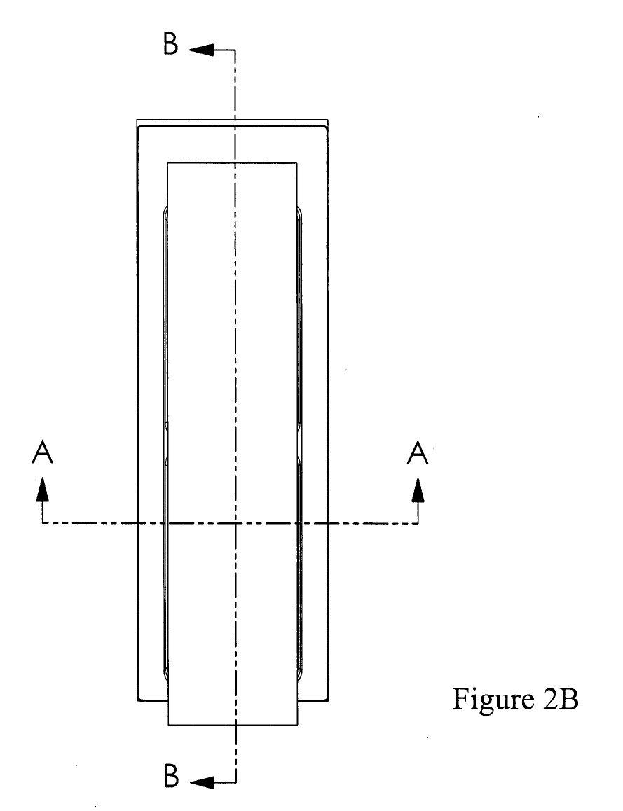

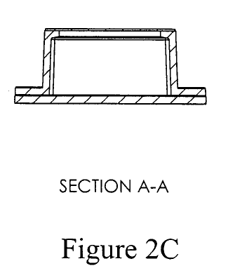

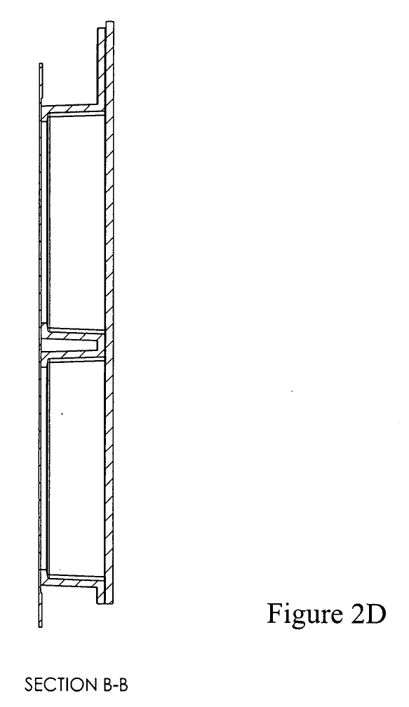

Another example is illustrated in FIG. 2, with several views displayed in FIGS. 2A through 2D. The two assay reaction system 200 has two assay chambers defined by a semi-rigid, thin-walled case 210, perimeter adhesive layer 230, and substrate 250 having arrays 261 and 262. The thin-walled case has two thin-walled 212 openings, a flat, thin bottom flange 214, and rims 216 at the upper end of the thin-walled openings. A top sealing strip 280 covers the opening of the thin-walled case to prevent evaporation of reaction contents. Also shown is a sheet 290 on top of the bottom flange of the case which carries a barcode. Not shown is a matching barcode on the bottom surface of the substrate.

The use of a thin-wall chamber design and polypropylene material (FIGS. 1 and 2) results in a chamber with substantially lower stiffness than the glass and prior art chambers. Also, for a given non-flatness, the lower stiffness puts less stress on the adhesive joint. The design also incorporates a flange for the majority of the width of the case to the glass (1.5 to 3.5 mm flange vs. <1 mm wall). The profile height of this flange is simply the thickness of the flange, about 0.9 mm. By comparison, the profile height of the chamber is about 8 mm. The greater profile height results in the chamber being substantially stiffer than the flange, but still substantially less stiff than prior art chambers. Even though the chamber is substantially stiffer than the flange, the break between chambers, consisting of the flange only, allows the 2-up chamber to conform to the glass without causing adhesion or scanning problems. Also, the open-top design reduces chamber stiffness. The opening in the top, through which fluid will be added and removed, has a flange of 2 mm width to allow a reliable seal to the sealing strip. The assay can be run in the described assembly by using an air gap and an orbital shaker to perform mixing.

This exemplary chamber designs are simpler than prior art chambers with clips, such as that shown in U.S. patent application Ser. No. 10/171,128. The plastic mold tooling is simpler and should cost less, and there is lower material usage. Polypropylene is a low-cost plastic and grades are readily available for medical applications. It does not require as tight tolerance control on the plastic. It eliminates the clip and its tight tolerance control.

This chamber lends itself easily to automation and semi-automation. This design enables scanning through the back of the glass with the chamber in place. In a preferred embodiment, the adhesive seal is manufactured in tape and reel form. For assembly, the chamber is placed on the adhesive while still on the tape. The chamber with adhesive may then be fed to a tape and reel placement machine to match the chamber with the glass.

Although a two-up chamber design is shown, this invention is not limited to the 2-up configuration. It may easily be extended to 1-up, 3-up and 4-up designs, with the flange break between chambers already described.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. At least one reaction chamber comprising:
(a) a substrate having a first surface and a second surface, wherein at least one reaction area is contained on said first surface;
(b) an adhesive layer having at least one perforation; and
(c) a case having at least one enclosed wall structures and a flat bottom flange, wherein each of said wall structures define a bottom opening, and a top portion opposite each bottom opening, whereas each top portion contains a top opening;
whereby said bottom flange of said case is attached to said first surface of said substrate through said adhesive layer so that each of said at least one reaction area, the at least one perforation and the at least one bottom opening are aligned and forms at least one individual reaction chamber.

2. The at least one reaction chamber of claim 1, wherein said top openings consist of small ports, and wherein the remaining parts of said top portions are enclosed.

3. The at least one reaction chamber of claim 1, wherein said enclosed wall structures have a substantial height above said substrate to allow air-interface mixing of reaction solutions.

4. The at least one reaction chamber of claim 1, wherein said case is made of rubber.

5. The at least one reaction chamber of claim 1, wherein said case having enclosed semi-rigid thin-wall structures, and a flat, thin bottom flange, wherein each of said top openings are substantially the same size as the corresponding bottom openings, and wherein the flexibility of the semi-rigid thin-wall and the thin bottom flange allows tight adhesion to said substrate and overcomes warp characteristics of said case or said substrate

6. The at least one reaction chamber of claim 5, wherein said thin-wall structures and said thin bottom flange of said case are of substantially the same thickness.

7. The at least one reaction chamber of claim 5, wherein said case is made of plastics.

8. The at least one reaction chamber of claim 5, wherein said case is made of polypropylene.

9. The at least one reaction chamber of claim 5, wherein each of said top openings contain a rim to allow easy and secure attachment of a sealing strip.

10. The at least one reaction chamber of claim 9, wherein the width of said rim is substantially twice the thickness of said thin-walls.

11. The at least one reaction chamber of claim 1, wherein said substrate is the same size of a standard microscopic slide.

12. The at least one reaction chamber of claim 11, wherein said substrate has two reaction areas and two individual reaction chambers are formed, each containing one of said two reaction areas.

13. The at least one reaction chamber of claim 11, wherein said substrate has four reaction areas and four individual reaction chambers are formed, each containing one of said four reaction areas.

14. The at least one reaction chamber of claim 11, wherein said substrate has eight reaction areas and eight individual reaction chambers are formed, each containing one of said eight reaction areas.

15. The at least one reaction chamber of claim 11, wherein said substrate has sixteen reaction areas and sixteen individual reaction chambers are formed, each containing one of said sixteen reaction areas.

16. The at least one reaction chamber of claim 1, wherein said substrate is the same size of a standard microtiter plate.

17. The at least one reaction chamber of claim 16, wherein said substrate has ninety-six reaction areas and ninety-six individual reaction chambers are formed in the pattern and dimension of a 96-well plate, each containing one of said 96 reaction areas.

18. The at least one reaction chamber of claim 1, wherein each of said at least one reaction area contains of a microarray of material selected from small molecules, biomolecules, cells and tissue samples.

19. The at least one reaction chamber of claim 18, wherein said biomolecules are selected from proteins, nucleic acids, and polysaccharides.

20. The at least one reaction chamber of claim 1, wherein said adhesive layer is a double-sided adhesive.

21. The at least one reaction chamber of claim 20, wherein said double-sided adhesive is pressure-sensitive.

22. The at least one reaction chamber of claim 1, further comprising an identifiable mark on top of said bottom flange of said case, or said second surface of said substrate in an area outside of said at least one microarray of materials.

23. The at least one reaction chamber of claim 22, wherein said identifiable mark is a barcode.

24. A reaction system comprising:
(a) a reaction chamber according to claim 1; and
(b) a top sealing strip.

25. A reaction system comprising:
(a) a reaction chamber according to claim 2; and
(b) sealing plugs for said small ports.

26. A method for preparing at least one reaction chamber comprising:
(a) providing a substrate having a first surface and a second surface, wherein at least one reaction area is contained on said first surface;
(b) providing an adhesive layer having at least one perforation;
(c) providing a case having at least one enclosed wall structure and a flat bottom flange, wherein each of said wall structures define a bottom opening, and a top portion opposite each bottom opening, whereas each top portion contains a top opening;
(d) adhering the case to a first face of the adhesive layer such that the at least one bottom opening is aligned with the at least one perforation; and
(e) adhering the first surface of the substrate to a second face of the adhesive layer such that the at least one reaction areas are aligned with the at least one perforations to form at least one individual reaction chambers.

27. A method for screening microarrays of materials comprising:
(a) preparing at least one reaction chamber according to claim 26, wherein each of said reaction areas contains a microarray;
(b) processing the microarray of material in the reaction chambers to acquire one or more desired characteristics of the microarray of material; and
(c) scanning the microarray of material to identify said characteristics.
